(19) United States
(12) Patent Application Publication
(10) Pub. No.: US 2021/0251570 A1
(43) Pub. Date: Aug. 19, 2021

(54) SURGICAL VIDEO CREATION SYSTEM

(71) Applicant: 3D MediVision Inc., Seoul (KR)

(72) Inventor: Kijin KIM, Seongnam-si (KR)

(73) Assignee: 3D MediVision Inc., Seoul (KR)

(21) Appl. No.: 17/246,490

(22) Filed: Apr. 30, 2021

Related U.S. Application Data
(63) Continuation of application No. PCT/KR2018/016633, filed on Dec. 26, 2018.

(30) Foreign Application Priority Data
Dec. 26, 2018 (KR) 10-2018-0168933

(51) Int. Cl. A61B 5/00; A61B 90/20; A61B 90/00

(52) U.S. Cl. CPC A61B 5/4887; A61B 90/20; A61B 90/37; A61B 2090/3941; A61B 90/39; A61B 2090/371; A61B 2090/373; A61B 5/0071

(57) ABSTRACT

Disclosed is a surgical video creation system, comprising: a surgical microscope comprising a light source; an image processing device configured so as to create a 3D video of surgical scenes and fluorescent images by means of the surgical scenes using the microscope; an optical adapter provided so that the image processing unit is mounted on the microscope; and a display unit configured so as to display the 3D video, wherein the image processing unit is configured so as to recognize the boundary of tumorous tissue by means of the fluorescent images and display the boundary in the 3D video, and the fluorescent images are created by light emitted from a fluorescent material which is selectively accumulated only in the tumor.

### CROSS-REFERENCE TO RELATED APPLICATIONS

The present application is a continuation of International Patent Application No. PCT/KR2018/016633, filed Dec. 26, 2018, which is based upon and claims the benefit of priority to Korean Patent Application No. 10-2018-0168933, filed on Dec. 26, 2018. The disclosures of the above-listed applications are hereby incorporated by reference herein in their entirety.

### TECHNICAL FIELD

The present invention relates to a surgical video creation system, and more particularly, to a system for creating a medical surgical stereoscopic video.

### BACKGROUND ART

Generally, a medical surgical microscope is a surgical device that can magnify the inside of a human body, which cannot be easily checked, during surgery. Such surgical microscopes are equipped with an imaging system that allows an operating doctor (hereinafter referred to as an “operator”) to see a surgical procedure through a monitor. However, such an imaging system displays two-dimensional (2D) images, and it is difficult to accurately observe and check a site subject to surgery using the 2D image, and thus there is a problem that the operator cannot perform surgery through the imaging system.

Also, the white balance adjustment function of a conventional imaging device has a limited range of adjustment and was developed based on sunlight. Therefore, in an environment in which a narrow and deep site is imaged using strong light such as a surgical microscope light source, even if the white balance is adjusted, distortion in which the color of human tissues or blood is expressed as pink instead of red occurs, and this causes problems in medical judgements related to, e.g., bleeding and lesions.

Also, there was developed a surgical method for removing a tumor using a microscope during surgery after a patient takes a special fluorescent substance in order to distinguish the tumor. Such fluorescent substances react with a patient's cancer cells to produce a unique substance, and the produced substance emits a fluorescent substance at an excitation wavelength, thereby distinguishing between normal tissues and tumors. However, in this surgical method, the fluorescent substance absorbed by the tumor cells can be visually distinguished only when lighting of a specific wavelength must be applied to an affected part while all lights in an operating room are turned off. Therefore, it is not possible to check tumors during surgery at any time while a conventional surgical lighting is turned on.

### Technical Problem

The present invention is directed to overcoming the above-described problems and to provide a stereoscopic video that can accurately show a surgical procedure.

The present invention is also directed to providing a stereoscopic video that can represent a red color without distortion in a surgical video using a medical surgical microscope.

Also, the present invention is also directed to providing a stereoscopic video that can distinguish normal tissues and tumors at any time without turning off the lighting of an operating room.

The technical objects to be achieved by the present invention are not limited to those mentioned above, and other technical objects, which are not mentioned herein, may be clearly understood by those skilled in the art from the following description.

### Technical Solution

According to an embodiment, there is provided a surgical video creation system including a surgical microscope including a light source, an image processing device configured to create a stereoscopic video of a surgical scene and a fluorescent image by means of the surgical scene using the microscope, an optical adapter configured so that the image processing unit is mounted on the microscope, and a display unit configured to display the stereoscopic video. The image processing unit is configured to recognize a boundary of a tumor tissue using the fluorescent image and mark the boundary in the stereoscopic video, and the fluorescent image is formed of light emitted from a fluorescent material which is selectively accumulated only in the tumor tissue.

Also, the image processing unit includes a filter configured to pass light corresponding to a first wavelength. The emitted light is light of the first wavelength. The fluorescent image is represented in a first color and a second color corresponding to the first wavelength. The image processing unit is configured to recognize a first region corresponding to the first color as the tumor tissue, recognize a second region corresponding to the second color as a normal tissue, and create the stereoscopic video in which a boundary between the first region and the second region is marked.

Also, the first wavelength is 635 nm, the first color is a red fluorescent color, and the second color is a blue fluorescent color.

Also, the image processing device is configured to mark the boundary by applying at least one of Sobel, Prewitt, Roberts, Compass, Laplacian, Laplacian of Gaussian (LoG) or Canny to the fluorescent image.

Also, the image processing device includes a mirror assembly configured to divide the image of the surgery scene into a first image and a second image and an image processing unit configured to create the stereoscopic video using the first image. The second image passes through the filter.

Also, the image processing unit is configured to measure a color temperature of the light source and interpolate chrominance of a red color of the first image using reference chrominance corresponding to the light source subject to the measurement.

Also, the light source is a light source with a color temperature between 3000 K and 7000 K.

The image processing device includes a camera configured to capture the surgery scene, a first stage configured to move the focus of the camera to the right or the left, and a second stage configured to move the focus of the camera upward or downward.

The first stage includes a first moving part and a first fixed part. The second stage includes a second moving part and a second fixed part. The first moving part is configured to move along an arc with respect to the first fixed part. The second moving part is configured to move along an arc with respect to the second fixed part.

The first stage includes a first knob. The second stage includes a second knob. The focus is moved to the right or left in response to rotation of the first knob and is moved up or down in response to rotation of the second knob.

The camera is fixed to the first moving part and the first stage is fixed to the second moving part, and the camera and the first stage are moved up or down in response to rotation of the second knob.

A stator including a horizontal surface and a vertical surface is between the first stage and the second stage. The first fixed part is fixed to the vertical surface, and the second fixed part is fixed to the horizontal surface.

### Advantageous Effects

According to an embodiment of the present invention, it is possible to provide a stereoscopic video that can accurately show a surgical procedure.

It is also possible to provide a stereoscopic video that can represent a red color without distortion in a surgical video using a medical surgical microscope.

It is also possible to provide a stereoscopic video that can distinguish normal tissues and tumors at any time without turning off the lighting of an operating room.

### DESCRIPTION OF DRAWINGS

FIG. 1 is a block diagram showing a configuration of a surgical video creation system according to an embodiment.

FIG. 2 is a configuration of a camera unit according to an embodiment.

FIG. 3 shows a tumor in a surgical video according to an embodiment.

FIG. 4 is a fluorescent image showing fluorescence emitted by the tumor of FIG. 3 reacting with a fluorescent substance.

FIG. 5 is a detection image in which the boundary of the tumor of FIG. 3 is marked in the surgical video according to an embodiment.

### DETAILED DESCRIPTION

Hereinafter, embodiments disclosed herein will be described in detail with reference to the accompanying drawings, but the same or similar elements are assigned the same or similar reference numerals, and redundant descriptions thereof will be omitted. Also, the accompanying drawings are only for making it easier to understand the embodiments disclosed herein, and therefore, the technical spirit disclosed herein is not limited by the accompanying drawings. Also, it should be understood that all modifications, equivalents, and alternatives falling within the spirit and scope of the invention are encompassed.

It will be understood that when an element is referred to as being “connected” or “coupled” to another element, it can be directly connected or coupled to the other element or an intervening element may be present. In contrast, when an element is referred to as being “directly connected” or “directly coupled” to another element, there are no intervening elements present.

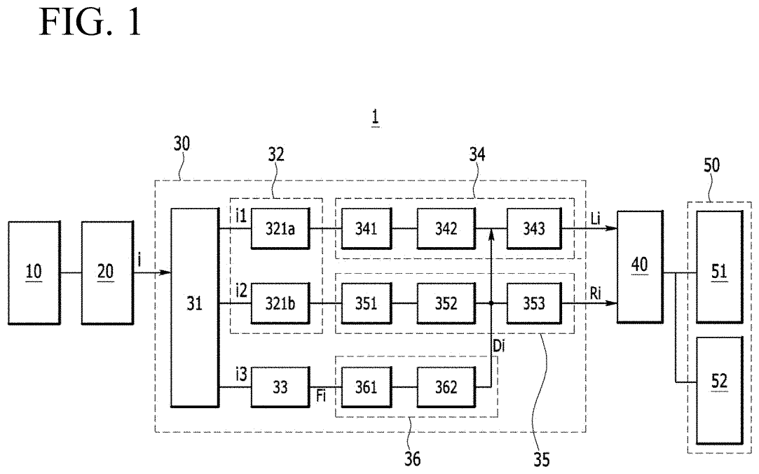

A surgical video creation system according to an embodiment will be described below with reference to FIG. 1. FIG. 1 is a block diagram showing a configuration of a surgical video creation system according to an embodiment.

Referring to FIG. 1, a surgical video creation system 1 according to an embodiment may include a surgical microscope 10, an optical adapter 20, an image processing device 30, a recorder 40, and a display unit 50 and may create a stereoscopic video including a surgical site and adjacent sites and a stereoscopic video with the boundary of a tumor being marked and then may display the videos in the display unit 50.

According to the present invention, prior to surgery, when a patient takes a fluorescent substance called 5-aminolevulinic acid (5-ALA; Gliolan), the active substance of the 5-ALA, protoporphyrin IX, is selectively accumulated only in tumor cells, and thus fluorescent light of a first wavelength (e.g., 635 nm) is emitted. This fluorescent light is brightest after a reference time (e.g., 2.5 hours after a patient takes 5-ALA).

Therefore, after the reference time, in a fluorescent image Fi seen through a filter unit 33, a tumor site is viewed in a first color of a first wavelength (e.g., red florescent color of 635 nm), and a normal tissue is viewed as a second color (e.g., blue fluorescent color). According to the present invention, the surgery may be a surgery to remove such a tumor, but the embodiments are not limited thereto.

The surgical microscope 10 is a motorized mechanical optical device used in various surgical operations and includes a light source (light-emitting diode (LED), Xeon, Halogen, etc.). An image of a surgical site or an adjacent site may be enlarged and viewed using the light of the light source. The color temperature of such a light source may be between 3000 K and 7000 K, but the present invention is not limited thereto.

The optical adapter 20 is configured such that the image processing device 30 may be mounted on the surgical microscope 10. The optical adapter 20 separates a surgical image i (hereinafter referred to as an image) input through the surgical microscope 10 into a plurality of images, and any one of the plurality of images is input to the image processing device 30.

The image processing device 30 includes a mirror assembly 31, a camera unit 32, a filter unit 33, a first image processing unit 34, a second image processing unit 35, and a third image processing unit 36. The image processing device 30 converts an image into a right-eye image Ri and a left-eye image Li and outputs the images in order to generate a stereoscopic image. Also, the image processing device 30 recognizes a patient's tumor using the fluorescent image Fi and combines an image in which the boundary of the recognized tumor is marked with the stereoscopic video.

The mirror assembly 31 may divide the image i into a plurality of images. Specifically, the mirror assembly 31 includes a plurality of reflectors (not shown) that horizontally and/or vertically reflect the image i. The mirror assembly 31 may separate the image i into a first image i1, a second image i2, and a third image i3 using the plurality of reflectors.

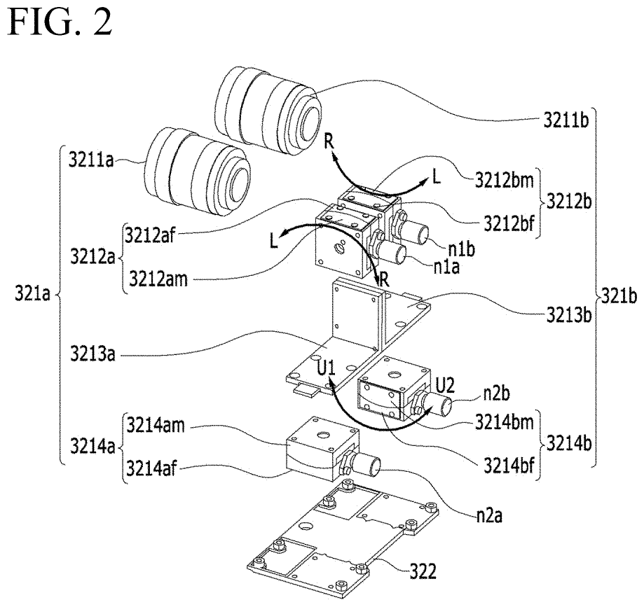

The camera unit 32 includes a first camera 321a, a second camera 321b, and a base plate 322 (see FIG. 2). The camera unit 32 captures a surgery scene using the surgical microscope 10. The camera unit 32 creates the first image it from the captured image and delivers the first image i1 to the first image processing unit 34. The camera unit 32 creates the second image i2 and delivers the second image i2 to the second image processing unit 35.

The first camera 321a includes a first camera 3211a, a first stage 3212a, a first stator 3213a, and a second stage 3214a.

The first camera 3211a captures the first image i1 and delivers the first image i1 to the first image processing unit 34.

The first stage 3212a includes a moving part 3214am, a fixed part 3212af, and a knob n1a, and the first camera 3211a is fixed to the moving part 3212am. The moving part 3212am moves along an arc to the right R or the left L according to the adjustment of the knob n1a, and the first camera 3211a moves to the right R or the left L in response to the movement of the moving part 3212am. Therefore, by adjusting the knob n1a, the focus of the first camera 3211a may be moved to the right R or the left L.

The first stator 3213a is in the shape of the letter “L.” The first stator 3213a is vertically symmetrical with the second stator 3213b and is in contact with the second stator 3213b on a symmetrical surface.

The second stage 3214a includes a moving part 3214am, a fixed part 3214af, and a knob n2a, and the bottom surface of the first stator 3213a is fixed onto the moving part 3214am. The moving part 3214am moves along an arc in one upward direction U1 or another upward direction U2 according to the adjustment of the knob n2a. Therefore, the first camera 3211a, the first stage 3212a, and the first stator 3213a move vertically in response to the movement of the moving part 3214am. That is, by manipulating the knob n2a, the focus of the first camera 3211a may be moved up or down.

The second camera 321b includes a second camera 3211b, a third stage 3212b, a second stator 3213b, and a fourth stage 3214b.

The second camera 3211b captures the second image i2 and delivers the second image i2 to the second image processing unit 35.

The third stage 3212b includes a moving part 3212bm, a fixed part 3212bf, and a knob n1b, and the second camera 3211b is fixed to the moving part 3212bm. The moving part 3212bm moves along an arc to the right R or the left L according to the adjustment of the knob n1b, and the second camera 3211b moves to the right R or the left L in response to the movement of the moving part 3212bm. Therefore, by adjusting the knob n1b, the focus of the second camera 3211b may be moved to the right R or the left L.

The second stator 3213b is in the shape of the letter “L.” The second stator 3213b is vertically symmetrical with the first stator 3213a and is in contact with the first stator 3213a on a symmetrical surface.

The fourth stage 3214b includes a moving part 3214bm, a fixed part 3214bf, and a knob n2b, and the bottom surface of the second stator 3213b is fixed onto the moving part 3214bm. The moving part 3214bm moves along an arc in one upward direction U1 or another upward direction U2 according to the adjustment of the knob n2b. Therefore, the first camera 3211b, the third stage 3212b, and the second stator 3213b move vertically in response to the movement of the moving part 3214bm. That is, by manipulating the knob n2b, the focus of the second camera 3211b may be moved up or down.

The fixed part 3214af and the fixed part 3214bf are fixed onto the base plate 322.

The filter unit 33 includes a band pass filter, and such a band pass filter passes light of a first wavelength in the third image i3 that is input. The third image i3 passes through the filter unit 33 and is converted into a fluorescent image Fi composed of light of the first wavelength, and the fluorescent image Fi is input to the third image processing unit 36.

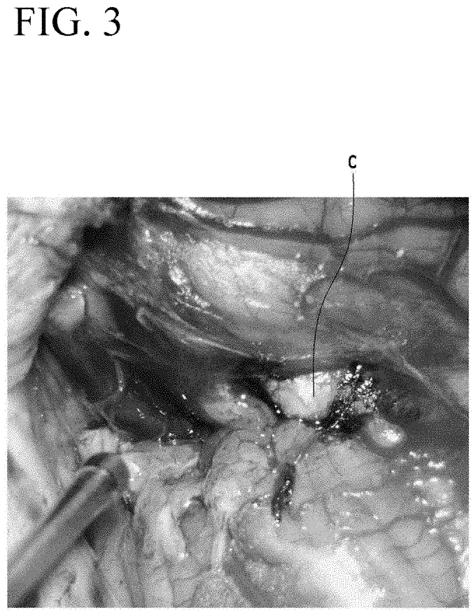

Specifically, when a patient takes 5-ALA, 5-ALA is absorbed only in the tumor (c) cell shown in FIG. 3 and is converted into a fluorescent substance (protoporphyrin IX), and the fluorescent substance emits fluorescent light of the first wavelength. In this case, the fluorescent substance emits the brightest light after a reference time.

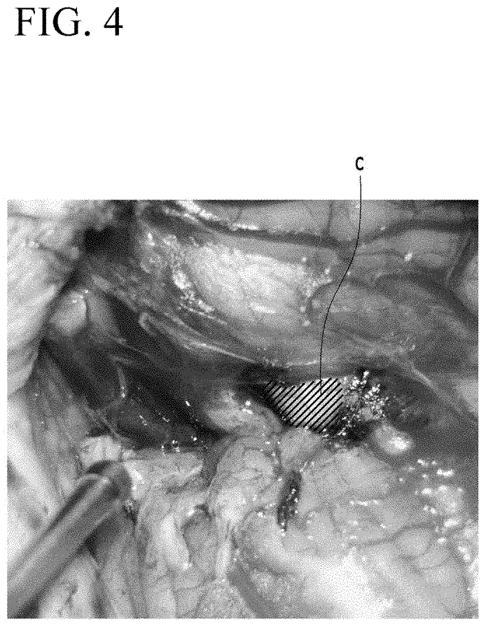

Therefore, the fluorescent image Fi is composed of a region of a first color and a region of a second color, and as shown in FIG. 4, the region of a tumor c, which is indicated by hatching, is expressed in the first color, and the region of a normal tissue other than the tumor c is expressed in the second color.

The first image processing unit 34 includes an image sensor 341, a processor 342, and an interpolation unit 343. The first image processing unit 34 detects information of a subject to be captured by the first camera 321a, generates an image signal, interpolates the generated image signal, and then overlaps a detection image Di of the third image processing unit 36 with the interpolated image to generate a left-eye image signal Li.

The image sensor 341 may be a charge-coupled device (CCD) or complementary metal-oxide-semiconductor (CMOS) sensor that detects information of a subject captured by the first camera 321a and generates an electric signal. However, the embodiments are not limited thereto.

The processor 342 generates an image signal using the electric signal generated by the image sensor 341. In this case, the processor 342 generates an image signal using the YCbCr color space composed of the luminance component Y and the chrominance components Cb and Cr. Image signals generated by the processor 342 are shown in Table 1 below.

TABLE 1

| Color | YCbCr |
|---|---|
| White | (235, 128, 128) |
| Yellow | (210, 16, 146) |
| Cyan | (170, 166, 16) |
| Green | (145, 54, 54) |
| Magenta | (106, 202, 222) |
| Red | (81, 90, 240) |
| Blue | (41, 240, 110) |
| Black | (16, 128, 128) |

The interpolation unit 343 measures the color temperature of the light source of the microscope 10 using the image created by the processor 342, adjusts the white balance, and interpolates chrominance corresponding to red family colors of the image signal with preset reference chrominance using only the chrominance components Cb and CR rather than the luminance component Y to create a left-eye image Li. At this time, the reference chrominance is chrominance that corresponds to a predetermined light source color temperature and in which red family colors can be expressed without distortion.

For example, referring to Table 2 below, the interpolation unit 343 adjusts the white balance of the image created by the processor 342 and then interpolates image chrominance corresponding to red family colors of the image created by the processor 342 with reference chrominance components Br and Rr corresponding to color temperature T.

TABLE 2

| Color Temperature | | |
|---|---|---|
| of Light Source | Cb | Cr |
| 3,000 K | −2 | −39 |
| . . . | . . . | . . . |
| T | Br | Rr |
| . . . | | . . . |
| 7,000 K | −49 | −17 |

Therefore, by interpolating the color chrominance of the red family colors of the image signal with the reference chrominance corresponding to the color temperature of the light source, a left-eye image Li that represents red color may be created with a constant chrominance component Cr regardless of the luminance of the light source of the microscope 10.

The second image processing unit 35 includes an image sensor 351, a processor 352, and an interpolation unit 353. The second image processing unit 35 detects information of a subject to be captured by the second camera 321b, generates an image signal, interpolates the generated image signal, and then overlaps a detection image Di of the third image processing unit 36 with the interpolated image to generate a right-eye image signal Ri.

The image sensor 351, the processor 352, and the interpolation unit 353 are substantially the same as the image sensor 341, the processor 342, and the interpolation unit 343, respectively, and thus a detailed description thereof will be omitted.

The third image processing unit 36 includes an image sensor 361 and a processor 362 and creates a detection image Di including a boundary between a tumor and a normal tissue using the fluorescent image Fi.

As described with reference to FIG. 4, the image sensor 361 may be a CCD or CMOS sensor that detects a fluorescent image Fi, in which a tumor c shown by hatching is represented in the first color and a normal tissue other than the tumor c is represented in the second color, and that generates an electric signal. However, the embodiments are not limited thereto.

The processor 362 recognizes a region corresponding to the first color as a tumor c using the electric signal generated by the image sensor 361, recognizes a region corresponding to the second color as a normal tissue, and creates a detection image Di including the boundary of the tumor.

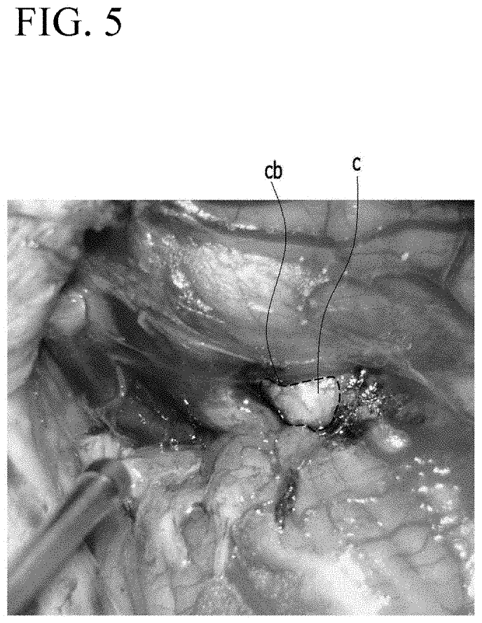

Specifically, referring to FIG. 5, the processor 362 recognizes the boundary between the first color and the second color as the boundary of the tumor c and creates a detection image Di including the boundary cb of the tumor c.

Also, the processor 362 may apply a video analysis algorithm to the video, recognize the region corresponding to the first color as the tumor c, recognize the region corresponding to the second color as a normal tissue, and create the detection image Di. The video analysis algorithm, which is an example, may distinguish the tumor c and the normal tissue using at least one of the boundary (edge) of the tumor c, the color of the tumor c, and the change in surface color spectrum of the tumor c, recognize the boundary between the tumor c and the normal tissue, and create the detection image Di. Also, the processor 362 may recognize the tumor c and the normal tissue by applying a deep learning technology to the video, but the embodiments are not limited thereto.

Also, the processor 362 may use at least one of Sobel, Prewitt, Roberts, Compass, Laplacian, Laplacian of Gaussian (LoG) or Canny to recognize the boundary between the tissue c and the normal tissue and create the detection image Di.

The recorder 40 stores the left-eye image Li and the right-eye image Ri.

The display unit 50 includes a plurality of monitors 51 and 52, and each of the plurality of monitors 51 and 52 displays a surgical image captured the left-eye image Li and the right-eye image Ri of the recorder 40 as a stereoscopic video.

In this way, a surgical site and even an adjacent site may be viewed as a stereoscopic video through the plurality of monitors 51 and 52, and thus an assistant as well as an operator perform surgery through the monitors 51 and 52 without performing surgery through the surgical microscope 10.

Also, conventionally, in order to check a fluorescent substance by selectively accumulating protoporphyrin IX in tumor tissues to emit light, it was possible to check tumors displayed as a fluorescent screen only by turning off the lighting of an operating room and using a microscope equipped with a filter. Also, such a fluorescent screen is composed of fluorescent substances and is represented in a color different from an original human tissue color.

However, according to embodiments, a stereoscopic video in which the boundary cb of a tumor is marked may be viewed through a plurality of monitors 51 and 52, and thus it is possible to distinguish a normal tissue and a tumor through the plurality of monitors 51 and 52 at any time during surgery without turning off the lighting of an operating room. Also, the stereoscopic video in which the boundary cb of the tumor is marked is displayed in a unique human tissue color rather than being displayed in a fluorescent screen.

Although the embodiments of the present invention have been described in detail above, the scope of the present invention is not limited thereto but encompasses various modifications and improvements made by those skilled in the art using the basic concept of the present invention defined in the appended claims. Therefore, in all respects, the detailed description above should not be construed as restrictive and should be considered as illustrative. The scope of the present invention should be determined by reasonable interpretation of the appended claims, and all changes within the equivalent scope of the present invention are included in the scope of the present invention.

### DESCRIPTION OF REFERENCE NUMERALS

1: Surgical video creation system

10: Surgical microscope

20: Optical adapter

30: Image processing device

40: Recorder

50: Display unit

1. A surgical video creation system comprising:
a surgical microscope comprising a light source;
an image processing device configured to create a stereoscopic video of a surgical scene and a fluorescent image by means of the surgical scene using the microscope;
an optical adapter configured so that the image processing unit is mounted on the microscope; and
a display unit configured to display the stereoscopic video,
wherein
the image processing unit is configured to recognize a boundary of a tumor tissue using the fluorescent image and mark the boundary in the stereoscopic video, and
the fluorescent image is formed of light emitted from a fluorescent material which is selectively accumulated only in the tumor tissue.

2. The surgical video creation system of claim 1, wherein
the image processing unit comprises a filter configured to pass light corresponding to a first wavelength,
the emitted light is light of the first wavelength,
the fluorescent image is represented in a first color and a second color corresponding to the first wavelength, and
the image processing unit is configured to recognize a first region corresponding to the first color as the tumor tissue, recognize a second region corresponding to the second color as a normal tissue, and create the stereoscopic video in which a boundary between the first region and the second region is marked.

3. The surgical video creation system of claim 2, wherein the first wavelength is 635 nm, the first color is a red fluorescent color, and the second color is a blue fluorescent color.

4. The surgical video creation system of claim 3, wherein the image processing device is configured to mark the boundary by applying at least one of Sobel, Prewitt, Roberts, Compass, Laplacian, Laplacian of Gaussian (LoG) or Canny to the fluorescent image.

5. The surgical video creation system of claim 4, wherein
the image processing device comprises:
a mirror assembly configured to divide the image of the surgery scene into a first image and a second image; and
an image processing unit configured to create the stereoscopic video using the first image, and
the second image passes through the filter.

6. The surgical video creation system of claim 5, wherein the image processing unit is configured to measure a color temperature of the light source and interpolate chrominance of a red color of the first image using reference chrominance corresponding to the light source subject to the measurement.

7. The surgical video creation system of claim 6, wherein the light source is a light source with a color temperature between 3000 K and 7000 K.

8. The surgical video creation system of claim 7, wherein the image processing device comprises:
a camera configured to capture the surgery scene;
a first stage configured to move a focus of the camera to the right or the left; and
a second stage configured to move the focus of the camera upward or downward.

9. The surgical video creation system of claim 8, wherein
the first stage comprises a first moving part and a first fixed part,
the second stage comprises a second moving part and a second fixed part,
the first moving part is configured to move along an arc with respect to the first fixed part, and
the second moving part is configured to move along an arc with respect to the second fixed part.

10. The surgical video creation system of claim 9, wherein
the first stage comprises a first knob,
the second stage comprises a second knob, and
the focus is moved to the right or left in response to rotation of the first knob and is moved up or down in response to rotation of the second knob.

11. The surgical video creation system of claim 10, wherein
the camera is fixed to the first moving part and the first stage is fixed to the second moving part, and
the camera and the first stage are moved up or down in response to rotation of the second knob.

12. The surgical video creation system of claim 11, wherein
a stator including a horizontal surface and a vertical surface is between the first stage and the second stage, and
the first fixed part is fixed to the vertical surface and the second fixed part is fixed to the horizontal surface.
